(19) United States
(12) Patent Application Publication
(10) Pub. No.: US 2015/0150549 A1
(43) Pub. Date: Jun. 4, 2015

(54) TEMPORARILY IMPLANTABLE TISSUE SEPARATOR

(71) Applicant: MAGEN ORTHOMED LTD, Ashkelon (IL)

(72) Inventors: Eran Hirszowicz, Ramat Gan (IL); Hamid Sharim, Cochav Yair (IL); Shmuel Beck, Giv'atayim (IL)

(21) Appl. No.: 14/413,798

(22) PCT Filed: Jul. 2, 2013
(86) PCT No.: PCT/IL2013/050562

§ 371 Date: Jan. 9, 2015

Related U.S. Application Data
(60) Provisional application No. 61/679,152, filed on Aug. 3, 2012.

(51) Int. Cl. A61B 17/02; A61C 8/00; A61F 2/02

(52) U.S. Cl. CPC A61B 17/0218; A61F 2/02; A61C 8/0092; A61B 2017/0023

(57) ABSTRACT

A temporarily implantable tissue separator comprises (a) a membrane having two substantially-smooth surfaces and (b) an extractor for withdrawing said membrane from the operation site after a healing period through a minimal surgical incision. The membrane is conformably placeable between tissues to be separated. The extractor has a distal end and a proximal end. The distal end of the extractor is continuously connected to the membrane and the proximal end remains loose outside the tissue.

### FIELD OF THE INVENTION

The present invention relates to an implant extractor, and, more specifically, to an extractor connected to a temporarily implantable membrane enabling easy withdrawal of the membrane-type tissue separator from the operation site after a healing period either percutaneously or through a minimal surgical incision.

### BACKGROUND OF THE INVENTION

A common clinical problem is inflammation of the rotator cuff tendons which can be intratendinous or peritendinous in nature. In addition shoulder trauma, especially during the healing phase of dislocated or separated shoulder or any conditions affecting shoulder motion (such as cerebrovascular accidents), is buildup of scar tissue or adhesions. Adhesion formation is a major clinical problem relating to consequences of treatment of inflammatory disease or any type of surgical repair. These adhesions might occur during the initial phases of the healing process after surgery or disease. Adhesions are a pathological state which involves the formation of abnormal tissue linkages. These linkages can, for example, impair bodily function, produce infertility, obstruct the intestines and other portions of the gastrointestinal tract (bowel obstruction) and produce general discomfort, e.g. pelvic pain. The condition can in some instances be life threatening. The most common form of adhesion occurs after surgery as a result of surgical interventions, although adhesion may occur as a result of other processes or events such as pelvic inflammatory disease, mechanical injury, radiation treatment and the presence of foreign material. Sometimes tissue irritation results in an inflammatory process. For example in the shoulder irritation of the rotator cuff tendons might result in a chronic inflammatory process leading to tears and limitation of joint motion.

To solve these problems, tissue seperators of various types have been developed. WO/2012/017438 discloses an apparatus and a method for a shoulder implant for the glenohumeral joint. The implant is expandable and deflatable during implantation. The implant is sized for the gelnoid fossa. The selection and/or implantation of the implant include adapting the implant by size and function to a particular patient's need.

U.S. Pat. No. 7,947,300 discloses thin resorbable lactide-polymer membranes. The thin membranes are constructed of polylactide-resorbable polymers, which are engineered to be absorbed into the body relatively slowly over time in order to reduce potential negative side effects. The membranes may be formed to possess an attribute of greatly reduced thickness, for example, a thickness of between about 0.010 mm and about 0.300 mm. The membranes can be extruded from polylactide polymers having a relatively high viscosity property, can be pre-shaped with relatively thick portions, and can be stored in sterile packages. It should be appreciated that the resorbable lactide polymer thin membrane is a heterogenous object which stays at the post-operative site for a long time. Then, resorption processes provide products of chemical decomposition which can be substantially detrimental for the patient.

Anti-adhesion membranes which are temporarily implantable into a site of a surgical operation or implanted as in a surgical procedure to separate tissues and prevent adhesions have been developed. There is however, a long felt and unmet need to provide suitable extractors and methods of extraction so as to facilitate withdrawal of tissue separators from the operative site, such that no traces were left after the membrane removal.

### SUMMARY OF THE INVENTION

It is hence one object of the invention to disclose an extractor for the removal of an anti-adhesion membrane arrangement being implantable into a site of a surgical operation for attenuating formation of post-surgical scar tissues and preventing tissue adhesion there between. The aforesaid arrangement comprises a membrane of a polymer-base material having two substantially-smooth sides. The aforesaid membrane is conformably placeable to maintain a smooth-surfaced barrier between operated and surrounding tissues for a period of healing to attenuate or eliminate any formation of scar tissues between operated and surrounding tissues. In some cases the membrane might possess an inflatable component that improves tissue separation. In other cases, the extractor or the membrane has a component preventing implant from dislodgement.

It is a core purpose of the invention to provide the arrangement comprising an elongated extractor connected to the membrane such that the membrane is withdrawable from the operative site after a healing period through a minimal surgical incision or percutaneously.

Another object of the invention is to disclose the extractor for facilitating an anti-adhesion membrane arrangement which is withdrawable through a surgical cannula.

A further object of the invention is to disclose the extractor adapted to withdraw from the body percutaneously a tissue separating membrane, to prevent adhesion formation in a prophylactic fashion or following physical or medical non-surgical adheosiolysis. In such implementation the membrane is spread to protect the relevant tissue preferably but not necessarily under imaging technique visualization. The membrane can later be retrieved by the extractor after a required period of implantation.

A further object of the invention is to disclose the extractor secured to the membrane by means of a funnel-like member having an expanded area for mechanical fixation of the wire to the membrane.

A further object of the invention is to disclose the extractor adapted to withdraw an anti-adhesion membrane arrangement for tissue separation implantable into a surgery site selected from the group consisting in a non-limiting manner of laminectomy, laminoplasty, joint surgery, ovary surgery, ligament, rotator cuff repair, carpal tunnel repair, nerve ending repair and muscle rupture treatment to prevent postsurgical fibrosis and adhesion.

A further object of the invention is to disclose the extractor which is disposable.

A further object of the invention is to disclose the extractor adapted to reside percutaneously during a healing period which could range from as short as 2 days to as long as 29 days or even longer.

A further object of the invention is to disclose the extractor and/or the tissue separator which is viewable by means of fluoroscopy or any other imaging technique (such as but not limited to sonography, nuclear magnetic resonance or computerized tomography)

A further object of the invention is to disclose the extractor and/or the tissue separator which is sterilizable.

A further object of the invention is to disclose the extractor and/or the membrane which is stitchable to the post-surgical site and adjacent surrounding tissues by bioresorbable suture materials.

A further object of the invention is to disclose the membrane or the extractor that are characterized also by clingability to biological tissues.

A further object of the invention is to disclose the membrane characterized by non-clingability to biological tissues.

A further object of the invention is to disclose the temporary implant reinforced for tissue shaping/scar modeling.

A further object of the invention is to disclose the temporary implant reinforced by at least one element selected from the group consisting of a spoke, a ridge, a notch, a groove, and an embedded wire/threat.

A further object of the invention is to disclose a method of extracting a membrane for attenuating formation of post-surgical scar tissues and preventing tissue adhesion therebetween. The aforesaid method comprises the steps of (a) providing a membrane assembly further comprising (i) a substantially planar membrane of a polymer base material having two substantially-smooth sides configured to maintain a smooth-surfaced barrier between operated and surrounding tissues for a period of healing to attenuate or eliminate any formation of scar tissues between operated and surrounding tissues; (ii) an extraction wire connected to the membrane such that the membrane is withdrawable from the operation site after a healing period through a minimal surgical incision; (i) inserting the assembly between operated and surrounding tissues; (ii) withdrawing the assembly after healing the operated tissues.

It is another core purpose of the invention to provide the step of withdrawing the assembly performed by means of extraction wire.

A further object of the invention is to disclose the step of either inserting or withdrawing the assembly performed though a surgical cannula.

A further object of the invention is to disclose the step of inserting the assembly and then stabilizing the membrane by stitching the assembly to the operated surrounding tissues by bioresorbable suture materials.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a schematic view of an embodiment of the present invention.

FIG. 2 is a schematic view of an embodiment of the present invention.

### DETAILED DESCRIPTION OF THE INVENTION

The following description is provided, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide an extractor for extracting from in situ an anti-adhesion membrane arrangement or a tissue separating membrane and a method of using the same.

An extractor is disclosed, useful for withdrawing an anti-adhesion tissue separating membrane arrangement implanted into a site of a surgical operation for attenuating formation of post-surgical scar tissues and preventing tissue adhesion therebetween is disclosed. The aforesaid arrangement comprises a substantially planar membrane of a polymer base material having two substantially-smooth sides configured to maintain a smooth-surfaced barrier between the operated and surrounding tissues for a period of healing to attenuate or eliminate any formation of scar tissues between the operated and surrounding tissues. An innovative feature of the arrangement lies in an extraction wire or thread connected to the membrane such that said membrane is withdrawable from the operation site after a healing period through a minimal surgical incision.

The term “scar tissue” hereinafter refers to areas of fibrous tissue (fibrosis) that replace normal tissue after injury or surgical operations. A scar tissue results from the biological process of wound or surgical operation repair in the skin and other tissues of the body. Thus, scarring is a natural part of the healing process. With the exception of very minor lesions, every wound (e.g. after accident, disease, or surgery) results in some degree of scarring.

An anti-scar membrane arrangement is implantable into a site of a surgical operation for attenuating formation of post-surgical scar tissues and preventing tissue adhesion therebetween, especially post-operative tissue adhesions. The aforesaid arrangement comprises a substantially planar membrane of a polymer base material having two substantially-smooth sides configured to maintain a smooth-surfaced barrier between the healing operated and the surrounding tissues for a period of healing to attenuate or eliminate any formation of scar tissues between the operated and surrounding tissues. In some embodiments of the present invention the membrane is acknowledged to have a clinging property to surrounding tissue akin to the “cling-film ” effect characterized by close adherence to the tissue.

In some embodiments of the present invention the membrane is acknowledged to have a non-clinging property, similar to the “cellophane effect” characterized by conforming to the tissue, yet not clinging to the tissue tightly.

The membrane is provided with an extraction wire connected to the membrane. The membrane is configured to be withdrawable from the operation or trauma site after a healing period.

According to an exemplary embodiment of the present invention, the wire is secured to the membrane by means of a funnel-like member having an expanded area for mechanical fixation of the wire to the membrane.

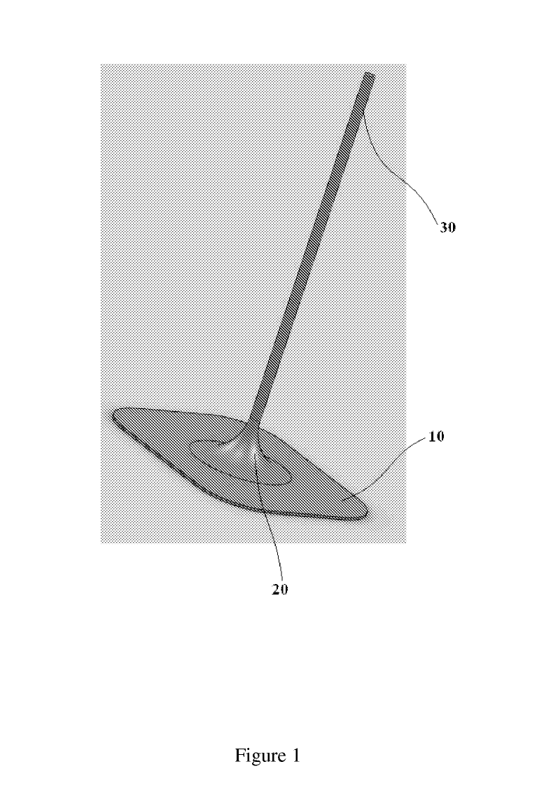

Reference is now made to FIG. 1, presenting a schematic view of an exemplary embodiment of the present invention. A substantially planar membrane 10 is made of a biocompatible and flexible sheet of 5-200 μm thickness with anti-adhesion properties to the tissue. The extractor 30 constitutes a polymer member, thread or wire of 0.5-3.0 mm diameter and 5.0-20.0 cm length. The membrane 10 can be characterized by a property of clingability to biological tissues, but non-clingability of the membrane 10 to biological tissues is also in the scope of the present invention. The membrane 10 can be made of Polyurethane, Teflon, Nylon, Cellophane or Silicone as well as various biodegradable polymers such as polylactide. Surfaces of the membrane 10 can be texturized to improve mechanical properties of the membrane surfaces. Mechanical connection of the wire 30 to the membrane 10 is reinforced by means of a funnel 20 having an expanded connection area with the membrane 10. The membrane shape can be conformed to subject's anatomy. The membrane 10 is placed between operated and surrounding tissues, and after the healing period the membrane 10 is withdrawn from the operation site (not shown) by means of pulling the wire 30. The withdrawal of the membrane 10 from the operation site can be performed both simply through an incision and through a cannula inserted into the incision. The cannula implanted into the patient's body is also in the scope of the invention.

It should be emphasized that the membrane 10 can be substantially reinforced such that a tissue during cicatrization takes a predetermined shape (tissue/scar modeling).

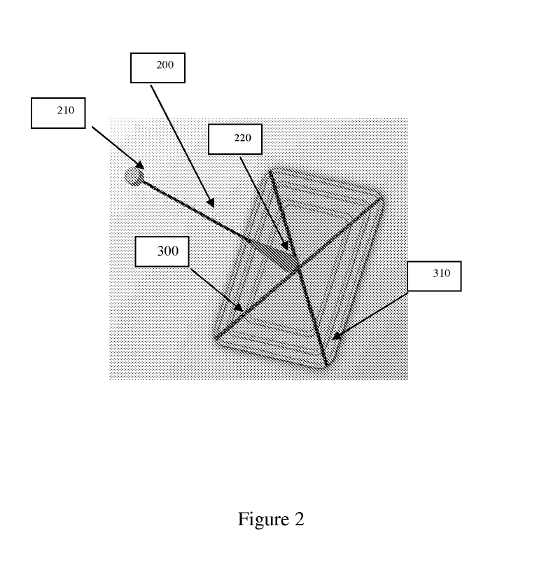

The temporary implant can be reinforced by at least one element selected from the group consisting of a spoke, a ridge, a notch, a grove, and an embedded wire/threat, Reference is now made to FIG. 2, presenting a schematic view of another exemplary embodiment of the present invention. In this embodiment, the extractor 200 has a proximal free or loose end with a 210 button, stopper, tab or similar, for grasping during removal. The distal end 220 is shaped in a non-uniform manner, in this case conical, to prevent cell migration to the junction between the extractor and the body of the tissue separator membrane. The proximal end of the extractor can be external or internal to the skin, according to the decision of the doctor or surgeon.

It is herein acknowledged that the extractor of the present invention can be used for extracting several embodiments of membrane type tissue separators, and also tissue separators with more complex designs such as the tissue separator of FIG. 2, 300, which has reinforcing members, ridges, spokes and other physical features.

It is herein acknowledged that some embodiments of the present invention have opening and closing means similar to an umbrella like arrangement.

It should be emphasized that use of the disclosed device for tissue modeling is in the scope of the present invention. In accordance with an exemplary embodiment of the present invention, the extractor anti-adhesion membrane arrangement is implantable into a site of laminectomy for prevention of post-laminectomy fibrosis and adhesion. The healing period is 29 days and longer. The assembly can be disposable, sterilizable and viewable by means of fluoroscopy or any other imaging technique. As needed, the assembly can be stitched to either operated or the surrounding tissues by suture materials that could be bioresorbable.

In accordance with the present invention, an extractor for withdrawing an anti-adhesion membrane arrangement implantable into a site of a surgical operation for attenuating formation of post-surgical scar tissues and preventing tissue adhesion therebetween is disclosed. The aforesaid membrane arrangement comprises a substantially planar membrane of a polymer base material having two substantially-smooth sides configured to maintain a smooth-surfaced barrier between the post-surgical site and adjacent surrounding tissues for a period of healing to attenuate or eliminate any formation of scar tissues between operated and surrounding tissues.

It is a core feature of the invention to provide the arrangement comprising an extractor connected to the membrane such that the membrane is withdrawable from the operation site after a healing period through a minimal surgical incision.

In accordance with one embodiment of the present invention, the extractor and anti-adhesion membrane arrangement is placeable or withdrawable though a surgical cannula.

In accordance with another embodiment of the present invention, the extractor is secured to the membrane by means of a funnel-like member having an expanded area for mechanical fixation of the extractor to the membrane.

In accordance with a further embodiment of the present invention, the extractor anti-adhesion membrane arrangement is implantable into a site of laminectomy for prevention of post-laminectomy fibrosis and adhesion.

In accordance with a further embodiment of the present invention, the extractor is disposable.

In accordance with a further embodiment of the present invention, the healing period is 29 days and longer.

In accordance with a further embodiment of the present invention, the extractor and assembly is viewable by means of fluoroscopy or other imaging technique.

In accordance with a further embodiment of the present invention, the extractor and membrane are sterilizable.

In accordance with a further embodiment of the present invention, the assembly might be sutured or otherwise attached to the post-surgical site and adjacent surrounding tissues by bioresorbable suture materials or tissue tacks or surface roughness.

In accordance with a further embodiment of the present invention, a method of attenuating formation of post-surgical scar tissues and preventing tissue adhesion therebetween is disclosed. The aforesaid method comprises the steps of (a) providing a membrane assembly further comprising (i) a substantially planar membrane of a polymer base material having two substantially-smooth sides configured to maintain a smooth-surfaced barrier between the post-surgical site and adjacent surrounding tissues for a period of healing to attenuate or eliminate any formation of scar tissues between operated and surrounding tissues; (ii) an extraction wire connected to the membrane such that the membrane is withdrawable from the operation site after a healing period through a minimal surgical incision; (i) inserting the assembly between operated and surrounding tissues; (ii) withdrawing the assembly after healing the operated tissues.

It is another core feature of the invention to provide the step of withdrawing the assembly performed by means of an extractor.

In accordance with a further embodiment of the present invention, the step of withdrawing the assembly is performed by pulling the extractor though a cannula.

In accordance with a further embodiment of the present invention, the step of withdrawing the assembly might comprise unstitching the assembly from the operated surrounding tissues by bioresorbable suture materials.

1-36. (canceled)

37. A temporarily implantable tissue separator comprising:
a. a membrane having two substantially-smooth surfaces; said membrane being conformably placeable between tissues to be separated; and
b. an extractor for withdrawing said membrane from the operation site after a healing period through a minimal surgical incision, said extractor has a distal end and a proximal end; said distal end is continuously connected to said membrane and said proximal end remains loose outside said tissue.

38. The temporarily implantable tissue separator of claim 37, wherein at least one of the following is true:
a. said extractor has a cone like distal end connected to said temporary implant and a substantially narrower proximal free end for grasping for withdrawing said membrane;
b. said extractor is withdrawable though a surgical cannula;
c. said extractor is secured to said membrane by means of a funnel-like member having an expanded area for mechanical fixation of a wire to said membrane; and
d. said membrane is inflatable.

39. The temporarily implantable tissue separator of claim 37, wherein at least one of the following is true:
a. said membrane is characterized by attachment profile to biological tissues;
b. said membrane is characterized by non-attachment to biological tissues;
c. said temporary implant is reinforced for tissue/scar modeling;
d. said temporary implant is reinforced by at least one element selected from the group consisting of a spoke, a ridge, a notch, a grove, and an embedded wire/thread.

40. The temporarily implantable tissue separator of claim 37, wherein at least one of the following is true:
a. said membrane is adapted for medical procedures selected from a group consisting of laminectomy, laminoplasty, scaffolding, fillers' design, plastic surgeries, tissue reconstruction, tissue construction, sinus lifting, sinus separation, craniotomy, carpal tunnel surgery, rotator cuff repair and any operation on a nerve, ligament, a tendon, a sinew, a string, or a cord;
b. said temporary implant is disposable;
c. said healing period is about 29 days and longer;
d. said separator is viewable by means of fluoroscopy;
e. said separator is sterilizable; and
f. said separator is stitchable to said operated and surrounding tissues by bioresorbable suture materials.

41. A temporary implant of the type used for separating tissues in a mammalian body in which a smooth sided membrane is conformably placeable between tissues for a period of healing;
wherein said implant comprises an extractor connected to the membrane to facilitate withdrawing said membrane from the operation site after a healing period through a minimal surgical incision.

42. The temporary implant of claim 41, wherein at least one of the following is true:
a. said extractor has a cone like distal end connected to said temporary implant and a substantially narrower proximal free end for grasping to facilitate withdrawing said membrane;
b. said proximal free end of said extractor is topped by a member selected from the group consisting of a button, stopper, tab or similar, for grasping during removal of the temporary implant;
c. said extractor is withdrawable though a surgical cannula; and
d. said extractor is secured to said membrane by means of a funnel-like member having an expanded area for mechanical fixation of said wire to said membrane.

43. The temporary implant of claim 41, wherein at least one of the following is true:
a. said membrane is adapted for medical procedures selected from a group consisting of laminectomy, laminoplasty, scaffolding, fillers' design, plastic surgeries, tissue reconstruction, tissue construction, sinus lifting, sinus separation, craniotomy, carpal tunnel surgery, rotator cuff repair and any operation on a nerve, ligament, a tendon, a sinew, a string, or a cord;
b. implant is disposable;
c. said healing period is about 29 days and longer; and
d. said implant is viewable by means of fluoroscopy.

44. The temporary implant of claim 41, wherein at least one of the following is true:
a. said implant is sterilizable; and
b. said implant is stitchable to said operated and surrounding tissues by bioresorbable suture materials.

45. An elongated extractor useful for withdrawing a temporary implant of the type used for separating tissues in a mammalian body in which a smooth sided membrane is located between tissues for a period of healing, said extractor having proximal and distal ends; wherein said extractor is connectable at the distal end to said membrane and the proximal end of said elongated extractor is free for grasping.

46. The elongated extractor of claim 45, wherein at least one of the following is true:
a. said extractor has a cone-like distal end connected to said temporary implant and a substantially narrower proximal free end for grasping to facilitate withdrawing said membrane;
b. said extractor is withdrawable though a surgical cannula.

47. The elongated extractor of claim 45, wherein at least one of the following is true:
a. said membrane is adapted for medical procedures selected from a group consisting of laminectomy, laminoplasty, scaffolding, fillers' design, plastic surgeries, tissue reconstruction, tissue construction, sinus lifting, sinus separation, craniotomy, carpal tunnel surgery, rotator cuff repair and any operation on a nerve, ligament, a tendon, a sinew, a string, or a cord;
b. said extractor is disposable; and
c. said healing period is about 29 days and longer.

48. The elongated extractor of claim 45, wherein at least one of the following is true:
a. said extractor is viewable by means of fluoroscopy;
b. said extractor is sterilizable; and
c. said extractor is stitchable to said operated and surrounding tissues by bioresorbable suture materials.

49. In a method of using a temporary implant for separating tissues in a mammalian body, said temporary implant having an extraction element, comprising the steps of:
a. providing temporarily implantable tissue separator comprising
i. a membrane having two substantially-smooth surfaces; said membrane being conformably placeable between tissues to be separated; and
ii. an extractor for withdrawing said membrane from the operation site after a healing period through a minimal surgical incision, said extractor has a distal end and a proximal end;
b. placing said implantable tissue separator such that said distal end is continuously connected to said membrane and said proximal end remains loose outside said tissue;
c. withdrawing said temporary implant from between said tissue, wherein said steps further comprise grasping the free proximal end of an extractor that is connected to said temporary implant and pulling it from between said tissues.

50. The method of claim 49, wherein at least one of the following is true:
a. said method comprises additional steps of withdrawing said temporary implant by drawing said free proximal end of said extractor through a cannula;
b. said device is inserted through a cannula;
c. said extractor allows anchorage of the device in the implantation site.

51. The method according to claim 49, wherein at least one of the following is true:
a. said step of withdrawing said assembly is performed though a surgical cannula;
b. said step of withdrawing said assembly is performed by said extractor secured to said membrane by means of a funnel-like member having an expanded area for mechanical fixation of said wire to said membrane;
c. said membrane is inserted into a surgical site selected from the group consisting of laminectomy, joint surgery, ovary surgery, ligament and muscle rupture treatment to prevent postsurgical fibrosis and adhesion.

52. The method according to claim 49, wherein a disposable tissue separator is provided.

53. The method according to claim 49, wherein said healing period is 29 days and longer.

54. The method according to claim 49, wherein said tissue separator is viewable by means of fluoroscopy.

55. The method according to claim 49, wherein a sterilizable tissue separator is provided.

56. The method according to claim 49, wherein said step of withdrawing said temporary implant comprises removing stitches connecting said membrane to said operated surrounding tissues.
